(19) United States
(12) Patent Application Publication
(10) Pub. No.: US 2015/0045692 A1
(43) Pub. Date: Feb. 12, 2015

(54) TEMPERATURE CONTROL MECHANISMS FOR STABLE SHAPE SENSING

(71) Applicant: KONINKLIJKE PHILIPS N.V., EINDHOVEN (NL)

(72) Inventors: Bharat Ramachandran, Morganville, NJ (US); Robert Manzke, Bonebuttel (DE); Raymond Chan, Andover, MA (US)

(21) Appl. No.: 14/387,597

(22) PCT Filed: Mar. 20, 2013
(86) PCT No.: PCT/IB2013/052196

§ 371 Date: Sep. 24, 2014

Related U.S. Application Data
(60) Provisional application No. 61/617,069, filed on Mar. 29, 2012.

(51) Int. Cl. A61B 5/06; A61B 5/01; A61B 5/107; G01L 1/24; G01K 13/00

(52) U.S. Cl. CPC A61B 5/065; G01L 1/242; G01K 13/00; A61B 5/1071; A61B 5/01
USPC 600/549; 600/587

(57) ABSTRACT

A fiber optic shape sensing system includes an elongated fiber optic shape sensing device (116) having a proximal region and a distal region. The distal region includes a first temperature at which shape sensing is performed. A temperature control device (102, 104, 106) is configured to control a second temperature at the proximal region of the shape sensing device to match the first temperature. The proximal region includes a launch region (118) for launching light into at least one optical fiber of the fiber optical shape sensing device.

This disclosure relates to medical instruments and more particularly to shape sensing optical fibers in medical applications having improved temperature control for temperature gradient regions.

Shape sensing based on fiber optics exploits the inherent backscatter in a conventional optical fiber. The principle involved makes use of distributed strain measurements in the optical fiber typically using characteristic Rayleigh backscatter patterns. A physical length and index of refraction of a fiber are intrinsically sensitive to environmental parameters, temperature, strain and, to a much lesser extent, pressure, humidity, electromagnetic fields, chemical exposure, etc. The wavelength shift, Δλ or frequency shift, Δν, of the backscatter pattern due to a temperature change, ΔT, or strain along the fiber axis, ∈ is:

$$Δλ / λ = - Δ v / v = {K}_{T} Δ T + {K}_{ɛ} ɛ , \\ where$$

$${K}_{ɛ} = 1 - \frac{{n}_{eff}^{2}}{2} ( {p}_{12} - v ( {p}_{11} + {p}_{12} ) ) .$$

The temperature coefficient K_{T} is a sum of the thermal expansion coefficient α=(1/Λ)(∂Λ/∂T) and the thermo-optic coefficient, ξ=(1/n)(∂n/∂T), with a typical value of 0.55×10-6° C.⁻¹ and a value of 6.1×10-6° C.⁻¹ for Germania-doped silica core fibers. The strain coefficient K∈ is a function of group index n, the components of the strain-optic tensor, pᵢⱼ and Poisson's ratio, μ. Typical values given for n, p₁₂, p₁₁ and μ for germanium-doped silica yield a value for K∈ of about 0.787. Thus, a shift in temperature or strain is merely a linear scaling (for moderate temperature and strain ranges) of the spectral frequency shift Δν. Naturally, this linear model would not apply if strains approach the elastic limit of the fiber, or temperatures approach the glass transition temperature of the fiber.

With a four or more core fiber system where one core is located in a center of the cross-section, strain due to bending and temperature effects can be separated out as long as no axial strain (tension) is applied, or in the event axial strain is present, the axial strain is known and controllable (so it can be calibrated out).

Optical shape sensing (OSS) is extremely sensitive to changes in ambient temperature. All shape sensing measurements are performed with reference to an initial launch point that needs to be fixed in position and orientation. A correlation with the reference measurement and tracking of phase shifts assume a fixed launch point. Shifts in the measured phase arise from changes in fiber geometry. An increase in the ambient temperature of even approximately 2° C. near the launch point can result in an expansion (or contraction) of the fiber that changes the effective launch point. This can cause loss of tracking and failure of OSS in an extreme case. For example, a 1.5 m fiber may have tens of thousands of data samples for a six degree of freedom measurement at 20 micron nodal increments. An expansion or compression of the fiber that results in multiple node shifts at the launch point can bring about a breakdown of shape tracking.

In accordance with the present principles, a fiber optic shape sensing system includes an elongated fiber optic shape sensing device having a proximal region and a distal region. The distal region includes a first temperature at which shape sensing is performed. A temperature control device is configured to control a second temperature at the proximal region of the shape sensing device to match the first temperature. The proximal region includes a launch region for launching light into at least one optical fiber of the fiber optical shape sensing device.

In one embodiment, a system for optical shape sensing system includes a processor, a memory coupled to the processor, and an elongated fiber optic shape sensing device having a proximal region and a distal region. The distal region includes a first temperature at which shape sensing is performed. A temperature controller is stored in the memory and configured to monitor and adjust a second temperature at a launch region for launching light into at least one optical fiber of the fiber optical shape sensing device. The launch region is in the proximal region of the shape sensing device. At least one temperature control element is responsive to the temperature controller and feedback from one or more temperature sensors to match the second temperature to the first temperature.

In another embodiment, a method for fiber optic shape sensing includes: providing an elongated fiber optic shape sensing device having a proximal region and a distal region, and a temperature control device located at the proximal region of the shape sensing device, the proximal region including a launch region where light is launched into at least one optical fiber of the fiber optical shape sensing device; measuring a first temperature at the distal region where shape sensing is performed; measuring a second temperature at the proximal region where the launch region is located; and controlling the second temperature to match the first temperature to provide an accurate launch region reference for optical shape sensing.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

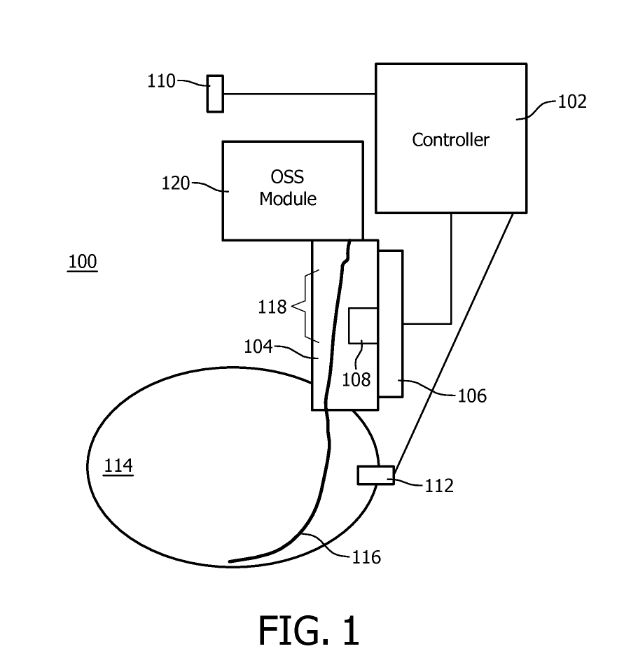

FIG. 1 is a block/flow diagram showing a shape sensing system which employs a temperature controlled region at a launch region to match a reference temperature in accordance with one embodiment;

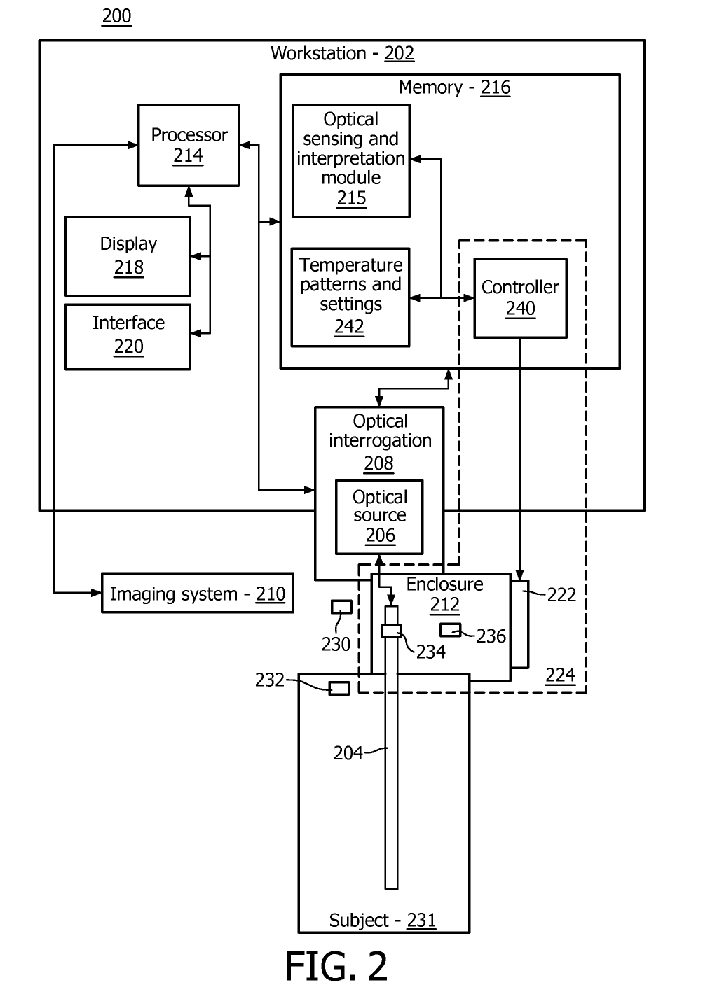

FIG. 2 is a block/flow diagram showing another shape sensing system which employs a workstation for controlling and monitoring temperature in a launch point region to match a reference temperature in accordance with one embodiment;

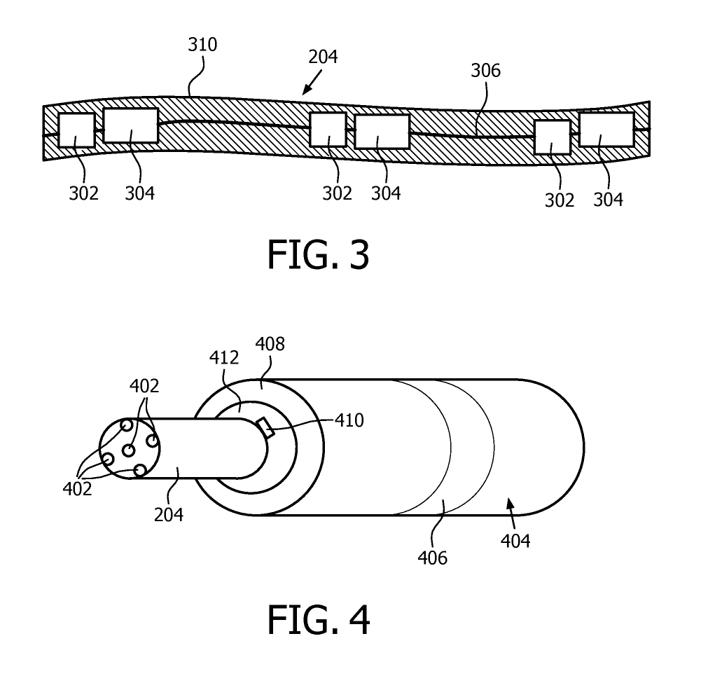

FIG. 3 is a cross-sectional view of a shape sensing device showing temperature sensors and temperature control devices integrated therein in accordance with one embodiment;

FIG. 4 is a perspective view of a jacketed shape sensing device showing temperature sensors and temperature control devices integrated therein in accordance with another embodiment; and

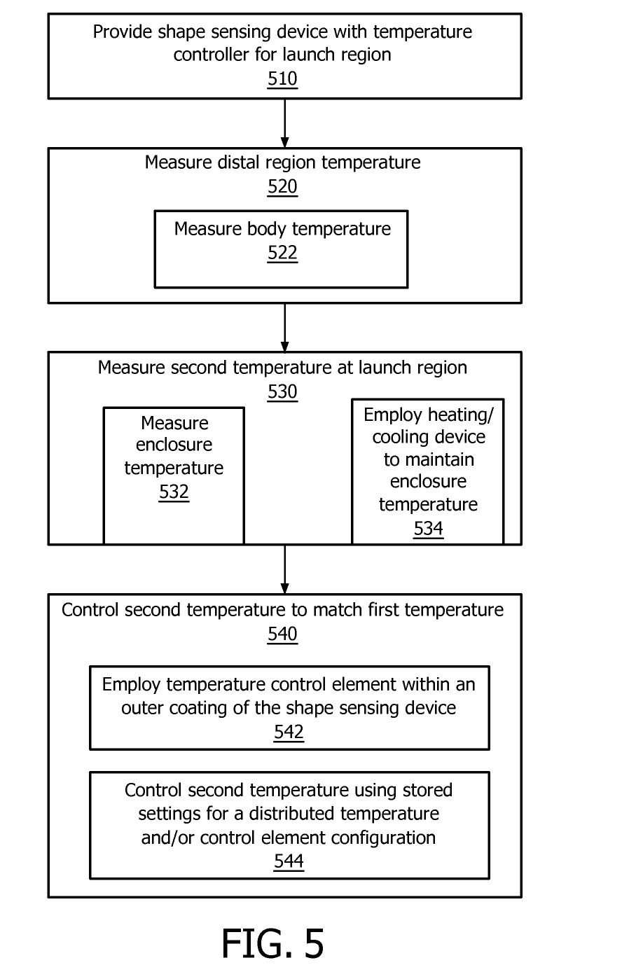

FIG. 5 is a block/flow diagram showing a method for shape sensing with a temperature controlled launch region in accordance with one embodiment.

In accordance with the present principles, systems and methods for controlling temperature in temperature gradient regions are provided. In a particularly useful embodiment, a system is provided to control the temperature around a launch region of a shape sensing fiber or fiber-enabled instrument. In one embodiment, the system includes a module and instruments for measuring reference temperature, a module and instruments for controlling temperature through heating or cooling and a processing unit that uses temperature measurements as input to provide control and feedback to maintain an equilibrium temperature. The present principles include different embodiments for a temperature control unit, such as, e.g., heating and/or cooling mats/pads, air-temperature control by air flow (introducing warm/cool air similar to a heater or an air-conditioner/refrigerator), water/fluid baths, surrounded by material that maintains temperature, reversible chemical reactions, etc.

Power-cycling an optical shape sensing (OSS) system may be performed to address temperature issues; however, this process requires recalibration and is impractical in a clinical setting. In addition, repeated correlation with a reference signal requires access to proprietary data that are usually inaccessible in the clinical setting. For example, dynamic correlation with a reference state, assumes that the signal from the reference state is available which is not always the case (this raw signal is usually proprietary and unavailable in a clinical setting). When only position and orientation information are available, the system and method for controlling the temperature of the shape sensing region near the launch region can be implemented in the clinical setting (e.g., an interventional room) without the need to correlate data or the need for extensive recalibration.

It should be understood that the present invention will be described in terms of medical instruments; however, the teachings of the present invention are much broader and are applicable to any fiber optic instruments. In some embodiments, the present principles are employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

Referring now to the drawings in which like numerals represent the same or similar elements and initially to FIG. 1, a system 100 for stabilization of an optical fiber launch region is shown in accordance with one illustrative embodiment. The system 100 includes a controller 102 that monitors temperatures in an interventional, clinical or other operative or testing environment. In one embodiment, a sensor 110 measures an ambient temperature in the environment. A sensor 112 measures a temperature of a subject 114. The subject may include a mechanical system or a patient's body. In one embodiment, the subject may include a model of a body for training or other purposes. A sensor or sensors 108 measure a feedback temperature employed to regulate a temperature of at least a portion of an optical shape sensing device 116.

The optical shape sensing device 116 is coupled to an optical shape sensing module 120 which provides a light signal and interprets a reflected light signal to determine a shape of the shape sensing device 116. The shape sensing device 116 passes into the subject 114 and, at a position where the device 116 exits the subject 114, an enclosure 104 is provided. The enclosure 104 provides a controlled environment in which the optical shape sensing device 116 is disposed during operation to prevent a temperature gradient between a portion of the optical shape sensing device 116 in the subject 114 and a portion of the optical shape sensing device 116 outside the subject. A launch region or point 118 on the optical shape sensing device 116 is included within the enclosure 104. The launch region 118 may include a portion of the fiber between an actual launch point and a fan out region for the light signal. This length can be as long as, e.g., 10 m, although other dimensions are contemplated. The launch region 118 could also be the region around a laser or the laser and a reference length of fiber (OSS module 120). Regardless of the scenario, any and all of these regions may be included in the launch region 118 and are regulated by a temperature control mechanism that is calibrated for optimal OSS performance.

The enclosure 104 may be actively temperature controlled using one or more temperature controlling methods. In one embodiment, the sensor 108 is employed to measure one or more of a temperature of the enclosure 104, a temperature of the media within the enclosure 104, a temperature of the optical shape sensing device 116, etc. A temperature control element 106 is controlled by the controller 102 to heat or cool the enclosure 104 in accordance with feedback from the sensor 108.

A change in ambient temperature near the launch region 118 would result in a loss of phase tracking which would result in unstable shape sensing that manifests as random jumps of shape and delivers inaccurate results. The system 100 makes shape sensing insensitive to fluctuations in ambient temperature. The ambient temperature induced shifts in optical backscatter patterns near the launch region 118 of the optical shape sensing device 116 are eliminated thereby delivering stable shape sensing.

In particularly useful embodiments, the sensors 108, 110 and 112 may include thermocouples, thermistors, optical fiber, etc. The temperature control element 106 may include heating and/or cooling mats or pads, resistance heaters, air-temperature control by air flow (e.g., heating ventilation and air-conditioning (HVAC) system), a heat exchanger, a water or other fluid bath, jacketing of the optical shape sensing device 116 (with a thermally insulating material that can maintain temperature passively or actively by electromagnetic means, chemical means (endothermic or exothermic chemical reactions), etc. In one embodiment, the optical shape sensing module 120 is included within the enclosure 104 to ensure appropriate temperature is maintained.

Referring to FIG. 2, a system 200 for stabilization of an optical fiber launch region is shown in accordance with another illustrative embodiment. System 200 may include a workstation or console 202 from which a procedure is supervised and/or managed. Workstation 202 preferably includes one or more processors 214 and memory 216 for storing programs and applications. Memory 216 may store an optical sensing and interpretation module 215 configured to interpret optical feedback signals from a shape sensing device or system 204. Optical sensing module 215 is configured to use the optical signal feedback (and any other feedback, e.g., electromagnetic (EM) tracking) to reconstruct deformations, deflections and other changes associated with a medical device or instrument and/or its surrounding region. The medical device may include a catheter, a guidewire, a probe, an endoscope, a robot, an electrode, a filter device, a balloon device, or other medical component, etc.

The shape sensing device 204 includes one or more optical fibers therein. The optical fibers connect to an optical interrogation module 208, which includes an optical source 206. The optical interrogation module 208 may be part of the workstation 202 or may be independent of the workstation 202. The optical source 206 provides light to the optical fibers of the shape sensing device 204. The optical source 206 may be included in the launch region in some embodiments. The optical sensing and interpretation module 215 may also be part of the workstation 202 or may be independent of the workstation 202. The module 215 interprets the shape sensing data collected by the optical interrogation module 208 and compares the data to known shapes to determine a present location of the shape sensing device 204 relative an image map.

Shape sensing device 204 with fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector.

A fundamental principle behind the operation of a fiber Bragg grating is Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors. In an FBG sensor, the measurand (e.g., strain) causes a shift in the Bragg wavelength.

One advantage of this technique is that various sensor elements can be distributed over the length of a fiber. Incorporating three or more cores with various sensors (gauges) along the length of a fiber that is embedded in a structure permits a three dimensional form of such a structure to be precisely determined, typically with better than 1 mm accuracy. Along the length of the fiber, at various positions, a multitude of FBG sensors can be located (e.g., 3 or more fiber sensing cores). From the strain measurement of each FBG, the curvature of the structure can be inferred at that position. From the multitude of measured positions, the total three-dimensional form is determined.

As an alternative to fiber-optic Bragg gratings, the inherent backscatter in conventional optical fiber can be exploited. One such approach is to use Rayleigh scatter in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, the 3D shape and dynamics of the surface of interest can be followed.

System 200 includes a temperature controller 240, which is stored in memory 216 and configured to receive temperature feedback from one or more temperature sensors or feedback control elements. The temperature sensors may include a sensor 230 that measures ambient temperature in a lab, clinic or other environment, a sensor 232 on a subject 231, a sensor 236 in an enclosure 212 and a sensor 234 on the shape sensing device 204. Different combinations of some or all of these or other sensors may be employed. In particularly useful embodiments, the sensors 230, 232, 234 and 236 may include thermocouples, thermistors, optical fibers, etc.

The sensors 230, 232, 234 and 236 provide feedback to the controller 240. The controller 240 controls one or more temperature control elements 222 in accordance with the temperature feedback. The temperature control elements 222 may include heating and/or cooling mats or pads, resistance heaters, air-temperature control by air flow, a heat exchanger, a water or other fluid bath, jacketing of the optical shape sensing device 204 (with a thermally insulating material that can maintain temperature passively or actively by electromagnetic means, chemical means (endothermic or exothermic chemical reactions), etc.

An imaging system 210 may be employed for in-situ imaging of a subject 231 during a procedure. The imaging system 210 may be employed by using data received from the shape sensing device 204 to image particular regions or register the image with the shape sensing data. Imaging system 210 may also be employed for collecting and processing operative or pre-operative images to map out a region of interest in the subject 231 to create an image volume for registration with shape sensing space. Imaging system 210 may include a fluoroscopy system, a computed tomography (CT) system, an ultrasonic system, a nuclear imaging system (e.g., positron emission tomography (PET), single photon emission computed tomography (SPECT)), etc.

Workstation 202 includes a display 218 for viewing internal images of the subject (patient) 231 including shape sensed images. Display 218 may also permit a user to interact with the workstation 202 and its components and functions, or any other element within the system 200. This is further facilitated by an interface 220 which may include a keyboard, mouse, a joystick, a haptic device, or any other peripheral or control to permit user feedback from and interaction with the workstation 202.

The enclosure 212 encloses a portion of the shape sensing device 204 that is subject to a temperature gradient. The enclosure 212 may include an enclosed volume of space where the shape sensing device 204 passes through. The enclosure 212 may include a jacket, sheath or covering that goes over the shape sensing device 204. In the case of the enclosed volume of space, the enclosure 212 may be filled with a medium, such as air, water, saline, etc. The volume is heated or cooled using temperature control elements 222. The volume may extend between at least the entry point into the subject 231 and at least a connection point for the optical source 206. In one embodiment, the optical source 206 represents the monitored launch region for the optical fibers of the shape sensing device 204. The launch region is included in the temperature controlled space of the enclosure 212.

In the case of the jacket for the enclosure 212, the jacket may be configured with temperature sensors (234) and temperature control elements 222 disposed along a length of the shape sensing device 204. The jacket may cover the shape sensing device 204 between the launch region and the subject 231.

A temperature control mechanism 224 generally includes the enclosure 212 (e.g., jacket, sheath or covering of the shape sensing device 204), at least one feedback sensor 234, the temperature control element(s) 222 and the controller 240. The mechanism 224 needs to provide a constant temperature wherever the launch region of the OSS fiber is. The temperature needs to match the temperature of the subject 231. Some examples of where the mechanism 224 may be positioned are described as follows. The temperature control mechanism 224 or a portion thereof can be provided within a laser console (optical source 206). The temperature control mechanism 224 or a portion thereof can be inserted within a small unit (enclosure 212) that may be fitted on/under an operating table, on a bed-side cart, on a detector of a C-arm (e.g., for an imaging system) or on or in equipment in an interventional room. The temperature control mechanism 224 or a portion thereof can be inserted within or in conjunction with devices like a ventilator, ECG monitor, etc., in the handle of the OSS enabled device (204), in a connector of the OSS enabled device, etc. In one embodiment, the temperature control mechanism 224 or a portion thereof may be worn by the subject 231 and be fixed on, under or over the body and employ the body temperature of the subject 231 as a temperature regulation mechanism.

The system 200 may be employed wherever OSS is employed. For example, the present principles may be employed in interventional X-ray suites for cardiology, or with other interventional systems and devices, etc.

Referring to FIG. 3 with continued reference to FIG. 2, a distributed system of temperature sensors 302 and temperature control elements 304 are integrated into the shape sensing device 204. In one embodiment, the sensors 302 are integrated within the shape sensing device 204. A plurality of sensors 302 may be employed where the temperature measurements are used as input to regulate and stabilize temperature at a fixed operating point across the shape sensing device 204. In this embodiment, the enclosure 212 may include a sheath or outer coating 310 of the shape sensing device 204, and the sensors 302 are included within the sheath 310 along with the temperature control elements 304. The sensors 302 may include electrical or optical elements to monitor temperature, and the temperature control elements 304 may be controlled accordingly using a controller (102 or 240). The temperature control elements 304 may include heating or cooling elements throughout the sheath corresponding to and controlled in accordance with the measured temperatures of the sensors 302. A dedicated optical fiber or fibers 306 (in addition to the shape sensing optical fibers) may be employed for temperature feedback in the shape sensing device 204. Note the heating/cooling is only needed for the portion of the shape sensing device 204 that is outside the subject 231. In this way, wireless interrogation of the external temperature measurement sensors may be made.

Since a distributed set of sensors 302 and elements 304 are employed, in one embodiment, the temperature controller 240 accesses optical shape sensing patterns 242 in an OSS performance library or look-up table to automatically determine target temperatures for optimal performance of the OSS system according to a desired quality control measure, e.g., RMS targeting error. In this way, the controller 240 can individually control each of the plurality of elements 304 with a power setting (or simply turning the elements on or off) in accordance with the pattern of measured temperatures to achieve a uniform temperature across the shape sensing device 204. The distributed temperature sensofirst r 302 measurements in the shape sensing device 204 are employed by the temperature controller 240 to equalize the distributed operating temperature of the shape sensing device throughout an operational length, e.g., matching a console or launch point temperature to a body temperature, in vivo.

Referring to FIG. 4, another distributed system of temperature sensors 410 and temperature control elements 406 are illustratively shown. FIG. 4 illustratively depicts a jacket 404 that fits over or may be manufactured onto the shape sensing device 204. The shape sensing device 204 may have many configurations including multiple optical fibers 402 disposed in a matrix. In one embodiment, the temperature sensor 410 is provided in the jacket 404 in contact with the shape sensing device 204. A thermally conductive sheath 412 is placed over the shape sensing device 204. The conductive sheath 412 may include a braided metal or other flexible or elastic conductive material. The jacket 404 includes an insulating layer 408 over the conductive sheath 402.

In one embodiment, the temperature of the shape sensing device 204 is measured by the temperature sensor 410 and compared to a reference temperature (e.g., a body temperature of a subject). The temperature control element 406 is activated to be heated or cooled to assist in matching the body temperature of the subject in the shape sensing device 204 that is external to the subject. The conductive sheath 412 ensures a relatively uniform temperature along the shape sensing device 204 due to its high conductivity. Likewise, the insulating layer 408 on jacket 404 distributes the heating/cooling load over a wider area and prevents hot or cold spots with high temperature gradient.

The temperature control elements (106, 222, 304, 406) described herein may take many forms. For example, in one embodiment, the temperature control elements 106, 222 may include air-flow control devices that regulate heat or cool in a limited space (e.g., fan heaters, a cooler or air conditioner) in an enclosure 104, 212. Air circulation can be employed to ensure that the entire region/volume has a similar temperature. In another embodiment, the temperature control can be provided using a water bath or other material (e.g., saline etc.), with the temperature of water maintaining the temperature of the volume of the enclosure 104, 212, wherein the entire unit is filled with water. Alternatively, temperature control elements 106, 222, 304, 406 may include an enclosed liquid cooling circulation mechanism that can be used with cooling channels or paths that are embedded in the jacket 404, sheath 310 or in the enclosure 104, 212. This may include plumbing tubes or channels. In one embodiment, the liquid control mechanism may use, instead of water, saline or any other fluid that still regulates temperature in a certain region.

In another embodiment, the temperature control elements 106, 222, 304, 406 include a reversible chemical reaction that occurs at a constant temperature, which can be used to maintain the temperature at the elements 106, 222, 304, 406 or in the enclosure 104, 212, while ensuring that the reaction does not influence shape sensing. In another embodiment, a chemical reaction (occurring over some time) can be used to maintain a constant temperature during the duration of an interventional procedure (which could be at ambient temperature, body temperature, or any other temperature during which the OSS system calibration is performed and while the system is operated to perform accurately and remain stable). In another embodiment, thermo-luminescence can be used to control and maintain temperature, e.g., a light bulb may generate heat when on in a space or enclosure 104, 212.

Referring to FIG. 5, a method for fiber optic shape sensing is shown in accordance with illustrative embodiments. In block 510, an elongated fiber optic shape sensing device having a proximal region and a distal region is provided. A temperature control device located at the proximal region of the shape sensing device is also provided. The proximal region includes a launch region where light is launched into at least one optical fiber of the fiber optical shape sensing device.

In block 520, a first temperature is measured at the distal region where shape sensing is performed. Measuring the first temperature may include measuring a body temperature of a subject in block 522. The body temperature is usually a stable reference point. In one embodiment, the body is employed to achieve the second temperature to match the first temperature by including the launch point or region of the optical fibers close or on the body of the subject.

In block 530, a second temperature is measured at the proximal region where the launch region is located. In block 532, measuring the second temperature may include measuring a temperature in an enclosure which receives at least the proximal region of the optical shape sensing device therein. In block 534, the temperature of the enclosure is adjusted to match the first temperature by using one or more of a chiller, a heater, an air conditioner, etc.

In block 540, the second temperature is controlled to match the first temperature to provide an accurate launch region reference for optical shape sensing. The enclosure may include an outer coating of the optical shape sensing device, and the second temperature may be measured within the outer coating. In block 542, the second temperature may be controlled using a temperature control element included within the outer coating.

In one embodiment, the optical shape sensing device includes a distribution of temperature sensors and the temperature control device includes at least one temperature control element configured to control the second temperature using feedback from the distribution of temperature sensors. In block 544, one or more patterns of a measured temperature distribution of the distribution of temperature sensors is correlated with power generation control settings for the at least one temperature control element to power at least one temperature control element. In another embodiment, a jacket is provided over the shape sensing device and the distribution of sensors and/or temperature controller elements are included in the jacket and controlled using a controller and/or with power generation control settings identified by using the patterns.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for temperature control mechanisms for stable shape sensing (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A fiber optic shape sensing system, comprising:
an elongated fiber optic shape sensing device having a proximal region and a distal region, the distal region having a first temperature in the distal region; and
a temperature control device configured to control a second temperature at the proximal region of the shape sensing device to match the first temperature, the proximal region including a launch region for launching light into at least one optical fiber of the fiber optical shape sensing device.

2. The system as recited in claim 1, wherein the temperature control device includes at least one temperature sensor configured to monitor the second temperature.

3. The system as recited in claim 1, wherein the temperature control device includes an enclosure for receiving the optical shape sensing device therein, such that a temperature in the enclosure includes the second temperature.

4. The system as recited in claim 3, wherein the enclosure includes a space and the space is temperature controlled using one or more of a chiller, a heater and an air conditioner.

5.-6. (canceled)

7. The system as recited in claim 3, wherein the enclosure includes an outer coating of the optical shape sensing device and the second temperature is measured within the outer coating.

8. The system as recited in claim 7, wherein the second temperature is controlled using a temperature control element included within the outer coating.

9. The system as recited in claim 1, wherein the temperature control device includes a controller configured to measure the first and second temperatures and control at least one temperature control element to match the first and second temperatures.

10. The system as recited in claim 1, wherein the optical shape sensing device includes a distribution of temperature sensors and the temperature control device includes at least one temperature control element configured to control the second temperature using feedback from the distribution of temperature sensors.

11. The system as recited in claim 1, wherein the temperature control device includes a jacket provided on the optical shape sensing device, the jacket including at least one temperature sensor and at least one temperature control element configured to control the second temperature using feedback from the at least one temperature sensor.

12. The system as recited in claim 11, wherein the jacket includes a conductive layer in contact with the optical shape sensing device and an insulating layer formed over the conductive layer and the at least one temperature control element being disposed on the insulating layer and the at least one temperature sensor being in contact with the optical shape sensing device.

13. A system for optical shape sensing system, comprising:
a processor;
a memory coupled to the processor;
an elongated fiber optic shape sensing device having a proximal region and a distal region, the distal region having a first temperature at which shape sensing is performed;
a temperature controller stored in the memory and configured to monitor and adjust a second temperature at a launch region for launching light into at least one optical fiber of the fiber optical shape sensing device, the launch region being in the proximal region of the shape sensing device; and
at least one temperature control element responsive to the temperature controller and feedback from one or more temperature sensors to match the second temperature to the first temperature.

14.-16. (canceled)

17. The system as recited in claim 13, wherein the optical shape sensing device includes a distribution of temperature sensors and the at least one temperature control element and is configured to control the second temperature using feedback from the distribution of temperature sensors.

18. The system as recited in claim 17, wherein the memory stores one or more patterns correlating a measured temperature distribution of the distribution of temperature sensors with power generation settings for the at least one temperature control element to power the at least one temperature control element.

19.-20. (canceled)

21. A method for fiber optic shape sensing, comprising:
providing an elongated fiber optic shape sensing device having a proximal region and a distal region, and a temperature control device located at the proximal region of the shape sensing device, the proximal region including a launch region where light is launched into at least one optical fiber of the fiber optical shape sensing device;
measuring a first temperature at the distal region where shape sensing is performed;
measuring a second temperature at the proximal region where the launch region is located; and
controlling the second temperature to match the first temperature to provide an accurate launch region reference for optical shape sensing.

22. The method as recited in claim 21, wherein measuring the first temperature includes measuring a body temperature of a subject.

23.-28. (canceled)
